(19) United States
(12) Patent Application Publication
(10) Pub. No.: US 2016/0059029 A1
(43) Pub. Date: Mar. 3, 2016

(54) TREATMENT APPARATUS WITH A POSITIONING DEVICE AND A STIMULATION DEVICE, AND STIMULATION METHOD

(71) Applicant: GMG Beratungs- und Beteiligungs Verwaltungs GmbH, Pullach (DE)

(72) Inventors: Elmar GRANDY, Oberschleissheim (DE); Klaus TEICHERT, Dover (GB)

(21) Appl. No.: 14/475,661

(22) Filed: Sep. 3, 2014

(51) Int. Cl. A61N 5/06; A61N 1/04; A61N 1/36

(52) U.S. Cl. CPC A61N 5/06; A61N 1/36014; A61N 1/0472; A61N 2005/067

(57) ABSTRACT

A positioning device (100), configured for positioning stimulation device (200), for low level laser therapy applications, on a person's body surface (1), includes adhering component (110), for accommodating stimulation device (200) and for a releasable arrangement on body surface (1), an antenna device (120), which is fixedly connected with adhering component (110), and is arranged for receiving control signals for controlling stimulation device (200), carrier contact section (130), which is fixedly connected with adhering component (110), and is arranged for providing an electrical contact with stimulation device (200), and switching device (140), which is fixedly connected with adhering component (110) and electrically interconnected between antenna device (120) and carrier contact section (130). Furthermore, treatment apparatus (300), configured for stimulation treatment of a person, includes positioning device (100) and stimulation device (200), a stimulation system, and a stimulation method are described.

### TECHNICAL FIELD

The present invention relates to a positioning device, which is adapted for positioning a stimulation device on a body surface of an organism, e. g. a person or animal to be treated. Furthermore, the present invention relates to a treatment apparatus comprising the positioning device and a stimulation device being coupled with the positioning device. Furthermore, the present invention relates to a stimulation system, comprising a plurality of treatment apparatuses and a main control. Furthermore, the present invention relates to a stimulation device, which is adapted for stimulating the body of an organism. Finally, the invention relates to a stimulation method for stimulating a body of an organism. Applications of the invention are available in particular in the field of stimulation treatments, using electrical and/or optical sources.

### TECHNICAL BACKGROUND

It is generally known to locally stimulate a body of a person to be treated by illuminating and/or heating the body surface with one or more light sources, e. g. laser diodes, or for improving wound healing. Light sources emitting ultraviolet, visible or infrared light are used for the exposure of biological tissue to radiation. For example, methods for exposing tissue to laser light have been developed which are known by the name of “Low Level Laser Therapy” (LLLT). LLLT methods are in particular based on stimulating materials-related processes in cells or tissue by the admission of light and/or heat (see e. g. U.S. Pat. No. 5,464,436).

Furthermore, the body can be stimulated by applying an electrical current. For example, DE 202 07 955 U1 discloses a stimulation device for electrically stimulating the body surface, wherein an electrical stimulation unit is adherently coupled with the body surface. The electrical stimulation unit is connected via a supply cable with a power supply. This technique has disadvantages if multiple electrical stimulation units are to be positioned on the body surface as the plurality of supply cables disturb the stimulation treatment. Furthermore, the supply cables have a certain weight, thus requiring a strong adherent connection between the electrical stimulation unit and the body surface. Again, the strong adherent connection causes an uncomfortable feeling for the person, thus impairing the stimulation result.

DE 101 28 629 A1 discloses a skin plaster, which carries a light source, a power supply and a control unit. The skin plaster is adapted for improving wound healing by optical stimulation. The skin plaster has an advantage as it does not require a connection with a main control via supply cables, thus allowing a comfortable treatment of the body surface. However, the skin plaster has a disadvantage in terms of controlling the operation thereof. As an example, if multiple skin plasters would be required, a synchronized switching of the light sources would not be possible with the conventional device. As a further disadvantage, the skin plaster of DE 101 28 629 A1 has a complex and expensive structure.

DE 10 2004 018 340 A1 disclose a treatment device for an external stimulation of a body, which is adapted for a wireless control. The treatment device comprises a stimulation device for an optical stimulation of the body surface and a positioning device for adherently positioning the stimulation device on the body surface. The positioning device includes an antenna, which receives control signals for controlling the stimulation device. The antenna is electrically connected with a control unit included in the stimulation device. Advantageously, a plurality of the conventional treatment devices can be simultaneously switched on using a radio control signal, which is received by the antennas and submitted to the control units of all treatment devices. Furthermore, different treatment devices can be controlled with different control signals, e.g. for applying specific stimulation programmes. However, the practical application of the conventional treatment device may suffer from the following disadvantages and restrictions. Firstly, there is an interest in minimizing the size of the treatment device for facilitating the adherent fixing thereof on the body surface. However, this is restricted due to the multiple components included in the stimulation device, like e.g. the control unit. Furthermore, the submission of the control signals received by the antenna via an electrical contact to the stimulation device is prone to be disturbed, e.g. by contact failures. As a further disadvantage, it has been found that an interference of the control signals of different treatment devices can occur. Finally, the structure and costs of manufacturing the conventional treatment device may result in restrictions for a mass application thereof.

### OBJECTIVE OF THE INVENTION

It is a first objective of the invention to provide an improved positioning device, which is adapted for positioning a stimulation device and which is capable of avoiding disadvantages of conventional techniques. In particular, it is the first objective of the invention to provide the positioning device with improved reliability of the electrical connection with the stimulation device and an improved reliability of adherently fixing the stimulation device on a body surface (e. g. skin of a person). It is a further objective of the invention to provide an improved treatment apparatus, in particular for LLLT applications, which is capable of avoiding disadvantages of conventional techniques and which in particular has an improved operation reliability. It is a further objective of the invention to provide an improved stimulation system comprising a plurality of treatment apparatuses. It is yet a further objective of the invention to provide an improved stimulation device, which is capable of stimulating a body of a person to be stimulated and which is capable of avoiding disadvantages of conventional techniques, in particular in terms of the structural complexity and manufacturing costs. Finally, it is another objective of the invention to provide an improved treatment method for stimulating a body, e. g. with light or electricity, wherein a plurality of the above treatment apparatuses are used.

These objectives are solved by a positioning device, a treatment apparatus, a stimulation system, a stimulation device and a treatment method of the invention.

### SUMMARY OF THE INVENTION

According to a first general aspect of the invention, the above objective is solved by a positioning device (holder, applicator), which is adapted for adherently fixing a stimulation device on a body surface of an organism, wherein the positioning device comprises an adhering component, an antenna device and a carrier contact section. The adhering component generally is adapted for accommodating the stimulation device in use and for a releasable arrangement with the stimulation device on the body surface. Preferably, the adhering component is a flexible carrier being capable of forming a receptacle, wherein the stimulation device can be arranged at the receptacle. The stimulation device can be hold by the adhering component, in particular in the adherently fixed condition thereof on a body surface. The antenna device is fixedly connected with the adhering component, e.g. it is integrated into the material of the adhering component or fixed to a surface thereof. The antenna device is adapted for receiving control signals from a main control for controlling the stimulation device, when it is coupled with the positioning device. The carrier contact section is a further portion of the adhering component. It is arranged such that an electrical contact is provided with the stimulation device when it is coupled with the positioning device.

According to the invention, the positioning device further comprises a switching device, which is fixedly connected with the adhering component. The switching device is electrically connected with the antenna device and the carrier contact section, and it is adapted for fulfilling a switching function in response to a control signal received by the antenna device. The switching device is electrically interconnected between the antenna device and the carrier contact section. Thus, contrary to the conventional technique, e.g. according to DE 10 2004 018 340 A1, switching signals are applied to the carrier contact section rather than radio frequency signals. The switching signals are less sensitive against contact failures or other wireless control signals. Furthermore, the provision of the switching device allows a simplified structure of the stimulation device. The size and weight of the stimulation device can be minimized, thus allowing a fixation on the body surface with increased reliability.

According to second general aspect of the invention, the above objective is solved by a treatment apparatus comprising the positioning device according to the above first general aspect of the invention and a stimulation device, in particular according to the fourth general aspect of the invention cited below, wherein the stimulation device is coupled with the positioning device such that a stimulation device contact section of the stimulation device is in electrical contact with the carrier contact section of the positioning device. Preferably, the stimulation device is detachably coupled with the positioning device, so that a separation of both components after use is possible.

According to a third general aspect of the invention, the above objective is solved by a stimulation system, which comprises a plurality of treatment apparatuses according to the second general aspect of the invention and a main control, which is configured for creating and transmitting control signals for controlling the stimulation devices of the treatment apparatuses, respectively.

According to a fourth general aspect of the invention, the above objective is solved by an stimulation device (in particular low level laser therapy device), which comprises at least one stimulation unit and a power supply section for supplying electrical power to the at least one stimulation unit. According to the invention, the stimulation device further comprises a stimulation device contact section, which is arranged, preferably exposed, on an outer surface of the stimulation device and which is adapted for receiving a switching signal. The stimulation device contact section is electrically connected with the at least one stimulation unit and the power supply section such that the at least one stimulation unit can be directly switched on or off in response to a switching signal applied to the stimulation device contact section, e. g. according to a predetermined stimulation pattern. Advantageously, the stimulation device according to the invention does not include a control unit for converting control signals received via an antenna into switching signals. Accordingly, the structure of the stimulation device is simplified and the size and weight thereof can be minimized.

Finally, according to a fifth general aspect of the invention, the above objective is solved with a treatment method for stimulating a body of a person to be stimulated, wherein the stimulation system according to the third general aspect of the invention is used.

According to a preferred embodiment of the invention, the adhering component of the inventive positioning device comprises at least one, preferably at least two adhesive layers, which are exposed for a contact with the body surface. Furthermore, the adhering component preferably comprises a carrier web, which is arranged adjacent to or between the one or more adhesive layer(s) wherein the carrier web carries the antenna device and the switching device and further is adapted for at least partially enclosing the stimulation device. Thus, in combination with the body surface, the carrier web is adapted to hold the stimulation device in use.

According to a particularly preferred embodiment of the invention, the antenna device and the switching device are arranged on an outer surface of the carrier web, while the carrier contact section is arranged and exposed on an inner side of the carrier web, i.e. on an inner side of the receptacle provided by the carrier web, facing to the stimulation device. Accordingly, the transmission of control signals to the antenna device is improved. Furthermore, an optical access to the switching device is allowed. If the switching device is adapted with an indicator light, an operation state of the switching device can be monitored by a user.

According to a further particularly preferred embodiment of the inventive positioning device, the antenna device, the switching device and the carrier contact section are arranged such that the distance of an electrical connection between the antenna device and the switching device is minimized. In particular, if the stimulation device is coupled with the positioning device, the length of the electrical connection between the antenna device and the switching device is shorter than the length of an electrical connection between the switching device via the carrier contact section to the stimulation device. Advantageously, the transmission of control signals via electrical connections within the positioning device, which could be deteriorated by other radio control signals, is minimized. Accordingly, the risk of interferences can be reduced.

According to a further preferred embodiment of the invention, the carrier contact section includes two contacts, which are to be connected with associated contacts of the stimulation device contact section. The switching device can be adapted for a pulsed operation, so that the stimulation device can be switched with pulse shaped switching signals, e. g. having frequencies in Hz- to kHz-ranges. Preferably, the switching device is adapted for electrically connecting the contacts in response to a control signal received by the antenna device. The contacts of the carrier contact section are short-circuited by the switching device in dependency on the control signal. In other words, the stimulation device can be directly switched with the switching device by closing an electrical circuit including the at least one stimulation unit and the power supply section. Alternatively, switching signals of the switching device can be submitted via the contacts for activating the stimulation device.

According to a further advantageous embodiment, the adhering component is a disposable component. The adhering component can be disposed as scrap material after use, while the stimulation device can be reused, in particular for a certain number of operation cycles, e.g. for 20 operation and recharging cycles. With a particularly preferred embodiment of the invention, the adhering component is an adhesive plaster with two adhesive layers with the carrier web there between. Advantageously, the inventive treatment apparatus including the stimulation device coupled with the positioning device simply can be applied to the body surface, like a conventional plaster.

According to a preferred embodiment of the inventive stimulation device, the at least one stimulation unit includes at least one of an optical stimulation unit and an electrical stimulation unit. Preferably, both of the optical and electrical stimulation units are included in the stimulation device. With a preferred treatment method, both of the optical and electrical stimulation units are driven simultaneously, so that the person feels the application of an electrical stimulation current simultaneously with the application of an optical stimulation.

According to a particularly preferred embodiment of the invention, the stimulation device is adapted for an output of a stimulation having the effect of Low Level Laser Therapy. Accordingly, the at least one stimulation unit includes at least the optical stimulation unit, including one or more laser diodes, emitting monochromatic light in the visible, near-infrared or infrared range, in particular in a range from 600 nm to 1000 nm. The optical stimulation unit is exposed on a surface of the stimulation device, so that it can be arranged in contact with a body surface. Preferably, the optical stimulation unit is adapted for an output power below 500 mW, in particular below 300 mW, e. g. 100 mW or lower, like 50 mW. The illuminated area of the surface to be treated is e. g. 1 cm² or smaller. Practical power densities are e. g. 25 mW/cm² or lower, like 10 mW/cm² or 5 mW/cm².

According to a further preferred embodiment of the inventive stimulation device, an enclosure (housing) is provided, which accommodates the at least one stimulation unit and the power supply. Preferably, the enclosure includes these components in a sealed, in particular humidity tight fashion. On one side of the enclosure (bottom surface), the at least one stimulation unit, in particular the optical stimulation unit and the electrical stimulation unit, is/are exposed towards the surrounding, while on a second, preferably opposite side of the enclosure (upper surface), the stimulation device contact section is arranged for a contact with the carrier contact section of the positioning device. Advantageously, the electrical connection between both contact sections reliably can be closed simply by the step of fixing the stimulation device with the positioning device on the body surface.

Further advantages of the invention are obtained if the stimulation device includes an anchoring section, which is adapted for a releasable connection with the positioning device. Advantageously, the anchoring section improves the reliability of the mechanical connection of the stimulation device with the positioning device and thus the reliability of the electrical connection there between. Preferably, the anchoring section comprises spikes and/or receptacle grooves, which are arranged for an engagement with the carrier web of the positioning device. Preferably, the spikes and/or receptacle grooves are provided on an outer side of the stimulation device enclosure.

Advantageously, the stimulation device contact section is adapted for fulfilling a double function. Firstly, it is electrically connected with the carrier contact section for the switching operation of the at least one stimulation unit. Secondly, the stimulation device contact section can be used for recharging the power supply section in the stimulation device. To this end, preferably the main control or an additional recharging device has at least one interface, preferably a plurality of interfaces, for electrically coupling one or more stimulation device(s) and recharging thereof. The recharging current can be supplied to the power supply via the at least one stimulation unit or, or it can be bypassed by a circuitry within the stimulation device.

### BRIEF DESCRIPTION OF THE DRAWINGS

Further advantages and details of the invention are described in the following with reference to the attached drawings, which show in:

FIG. 1: perspective views of a preferred embodiment of a positioning device according to the invention;

FIG. 2: a side view of a preferred embodiment of a treatment apparatus according to the invention;

FIG. 3: various outer views of an embodiment of the stimulation device according to the invention;

FIG. 4: various views of a further embodiment of a stimulation device according to the invention;

FIG. 5: illustrations of the internal structure of an embodiment of the stimulation device according to the invention;

FIG. 6: an example of a main control of a stimulation system according to the invention; and

FIG. 7: an example of a recharging device of a stimulation system according to the invention.

### PREFERRED EMBODIMENTS OF THE INVENTION

Embodiments of the invention are described in the following with particular reference to the application of a stimulation device including both an electrical stimulation unit and an optical stimulation unit. It is emphasized that the implementation of the invention is not restricted to the illustrated embodiments but rather possible also with stimulation devices including an electrical stimulation unit or an optical stimulation unit only. Details of stimulation methods, in particular treatment procedures with predetermined electrical and/or optical stimulation patterns comprising pulse stimulation currents and/or pulsed stimulation illumination are not described as they are known as such from prior art. Preferably, LLLT treatment procedures are applied, wherein the optical stimulation patterns is switched according to a known LLLT protocol. If the optical and electrical stimulation are combined, both of the optical and electrical stimulation units preferably are operated in synchronized fashion, i. e. they are simultaneously switched on/off. The drawings show schematic illustrations only. In practice, the details of the illustrated components can be modified and adapted to the requirements of the practical use.

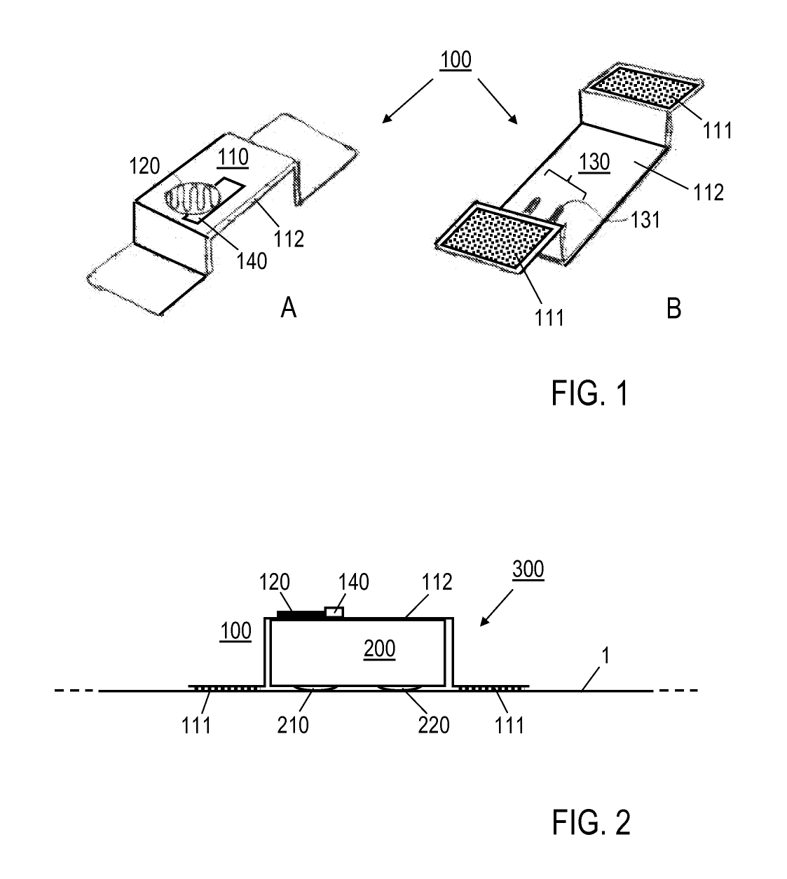

FIG. 1 schematically illustrates a preferred embodiment of an inventive positioning device 100, comprising the adhering component 110, the antenna device 120, the carrier contact section 130 and the switching device 140. FIG. 1A shows an upper perspective view of the positioning device 100, while FIG. 1B illustrates the corresponding perspective view from below.

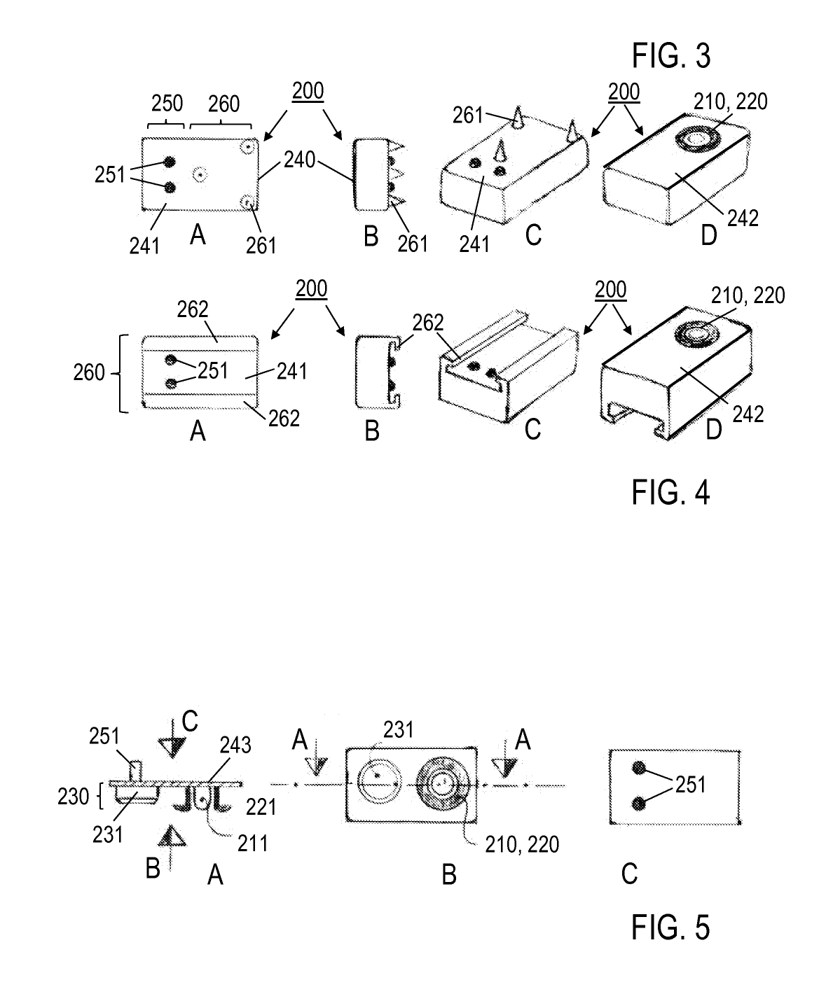

The adherent component 110 comprises a strip, which is made of a flexible material, e.g. a textile material and/or a plastics material. With a practical example, the strip has a rectangular shape with a size of e.g. 1 cm*5 cm. FIGS. 1A and 1B illustrate the positioning device in a folded condition, thus emphasizing a receptacle formed by the strip for accommodating an stimulation device, e.g. according to FIG. 3 or 4. Alternatively, without the coupling with the stimulation device, the strip of the adhering component 110 can have a plane shape. As a further alternative, the folded shape (as shown) can be created by the structure of the strip, e.g. by the provision of appropriate folding sections perpendicular to the longitudinal extension of the strip.

At the longitudinal ends of the adhering component 110, two wings are provided, each carrying an adhesive layer 111. The adhesive layers are exposed for a connection with a body surface 1 (see FIG. 2). Furthermore, a central section of the strip provides a carrier web 112, which forms the receptacle for accommodating the stimulation device.

The antenna device 120 and the switching device 140 are arranged on an outer surface of the adhering component 110, in particular on the carrier web 112. Alternatively, they could be arranged on one of the wings carrying the adhesive layers 111. The antenna device 120 and the switching device 140 may comprise separate components, wherein the antenna device 120 comprises an antenna for receiving radio frequency control signals as it is known as such and the switching device 140 comprises a circuit for creating switching signals in response to the control signals, in particular for creating a short-circuit between the contacts 131 of the carrier contact section 130. Alternatively, both of the antenna device 120 and the switching device 140 are provided as a common component, e. g. as a transponder device including an antenna and an integrated circuit for fulfilling the switching function of the switching device 140.

The carrier contact section 130 comprises two contacts 131, being formed as straight strips on the inner side of the carrier web 112. The contacts 131 are directly connected with the switching device 140. The contacts 131 comprise e.g. Cu with a length of e.g. 5 mm and a width of e.g. 1 mm.

FIG. 2 schematically shows a side view of an embodiment of the inventive treatment apparatus 300 comprising the positioning device 100 as shown in FIG. 1 and the stimulation device 200, which is further described below with reference to FIGS. 3 to 5. The adhering component of the positioning device 100 is connected via the adhesive layers 111 with the body surface 1 of a person to be stimulated. On the upper side of the carrier web 112 of the adhering component, the antenna device 120 and the switching device 140 are arranged, e. g. side by side, while the stimulation device 200 is accommodated by the receptacle formed by the carrier web 112. With the fixed condition, the electrical stimulation unit 210 and the optical stimulation unit 220 point towards the body surface 1. Activating the stimulation units 210, 220 with the switching device 140 results in a stimulation of the body according to a predetermined stimulation protocol.

FIGS. 3A to 3D illustrate various outer views of a first embodiment of the stimulation device 200. The stimulation device 200 comprises an enclosure 240 having an upper surface 241 (FIG. 3A) and a bottom surface 242 (FIG. 3D). The enclosure 240 is made of e.g. plastic or steel. Preferably, the enclosure 240 is sealed at all sides thereof so that an introduction of humidity or liquid water into the inner space of the enclosure 240 is prevented. The upper surface 241 includes the stimulation device contact section 250 with two contacts 251 being exposed to the carrier contact section 130 (see FIG. 1B). Furthermore, the upper surface 241 carries an anchoring section 260 comprising three spikes 261 (see FIGS. 3B, 3C), which protrude from the upper surface 241. When the stimulation device 200 is coupled with the positioning device 100, spikes 261 are introduced into the carrier web 112 for a fixation of the stimulation device at the positioning device 100.

On the bottom surface 242, the electrical and optical stimulation units 210, 220 are exposed. With the embodiment illustrated in FIG. 3D, both stimulation units have a concentric arrangement with an inner optical stimulation unit 210, e.g. a light emitting diode or a laser diode 211, and an outer stimulation electrode 221 (see FIG. 5A). Alternatively, multiple laser diodes 211 and/or multiple stimulation electrodes 221 can be provided at a stimulation device 200.

FIG. 4 shows outer views of a further embodiment of the stimulation device 200, in particular with the upper surface 241 and the bottom surface 242. The upper surface 241 includes the contacts 251 and an anchoring section 260 with two receptacle grooves 262 (see FIGS. 4B, 4C). The receptacle grooves 262 are configured for an accommodation of the edges of the carrier web 112 (see FIGS. 1, 2).

FIG. 5 shows internal views of the stimulation device 200. According to FIG. 5A, a support plate 243, like e.g. a printed circuit board, is arranged in the enclosure 240 (not shown in FIG. 5). On a first side of the support plate 243, facing to the bottom surface of the enclosure, the electrical and optical stimulation units 210, 220, with the light emitting diode 211 and the stimulation electrode 221 are arranged. Furthermore, the support plate 243 carries a power supply section 230, comprising e.g. a rechargeable accumulator. On the opposite site of the support plate 243, the electrical contacts 251 are arranged, which are exposed at the upper surface 241 of the enclosure 240 (see FIGS. 3A, 4A).

FIG. 5B shows the plan view of the support plate 243 according to arrow B in FIG. 5A. Accordingly, the support plate 243 carries the accumulator 231 and the stimulation units 210, 220. Furthermore, FIG. 5C shows a plan view of the support plates 243 according to arrow C in FIG. 5A, including the contacts 251.

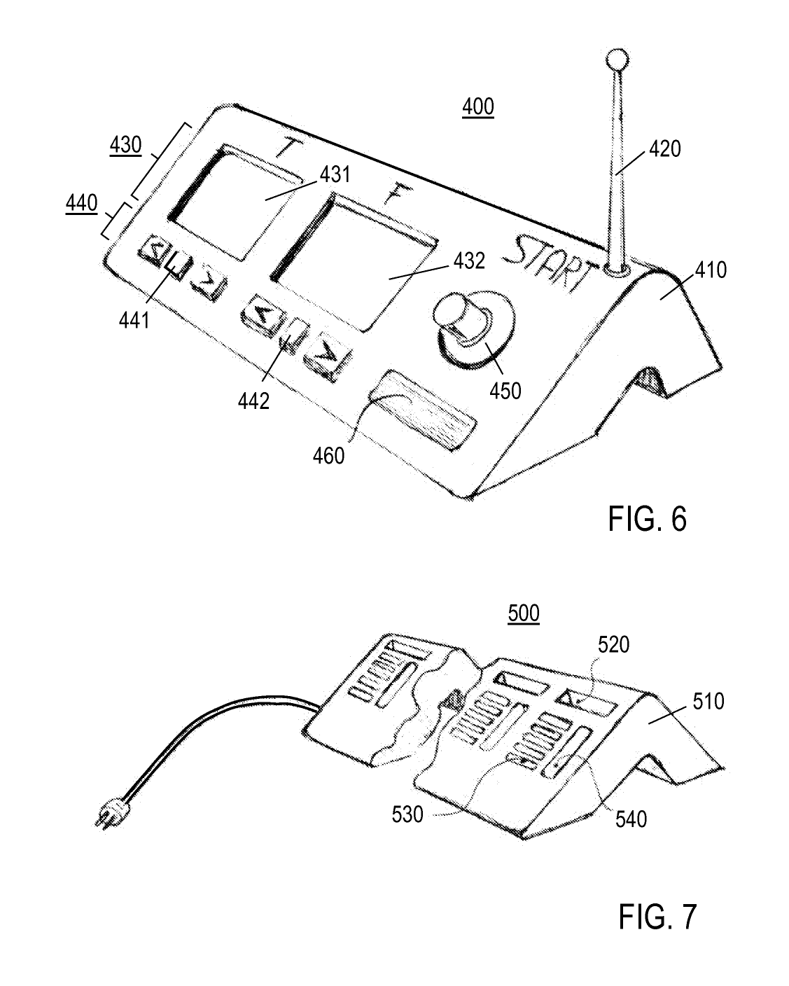

FIGS. 6 and 7 illustrate a main control 400 and a recharging device 500, which can be provided as separate apparatuses (as shown) or alternatively integrated into a common apparatus. The main control 400 of FIG. 6 comprises a housing 410, a transmitter antenna 420, a display device 430 with a first display 431 and a second display 432, a selector device 440 with a first selector 441 and a second selector 442, a start switch 450 and an acoustic alarm device 460. The first display 431 shows a time program of the stimulation protocol selected with the first selector 441, and the second display 432 shows a frequency program of the stimulation protocol selected with the second selector 442. The acoustic alarm device 460 can be used for indicating the end of the stimulation procedure.

The charging device 500 of FIG. 7 (partially shown) comprises a housing 510, a plurality of interface sections 520 (e. g. 8 or 12), a plurality of associated charging condition display devices 530 and a plurality of associated alarm display devices 540 (each for one of the interface sections 520). The interface sections 520 are adapted for electrically coupling and recharging stimulation devices 200. Preferably, the interfaces 520 have a shape being adapted to the outer shape of the enclosure 240 of the stimulation device 200. The charging can be implemented directly using the contacts 251 of the stimulation device 200 (see e.g. FIGS. 3A, 4A).

In practical use, an inventive stimulation system includes a plurality of treatment apparatuses 300 (according to FIG. 2) and a main control 400, optionally with the integrated recharging device 500. The treatment apparatuses 300 are distributed and adherently fixed in a detachable manner on the body surface of a person to be treated. The time and frequency programme of the stimulation procedure is selected by a user with the first and second selectors 441, 442 of the main control 400. The operation of the treatment apparatuses 300 is activated by activating the start switch 450. Control signals are transmitted via the transmitter antenna 420 to the antenna devices 120 of the treatment apparatuses 300. The switching devices 140 close the contacts 251 of the stimulation devices 200 with the parameters of the selected time and frequency programs, thus starting the operation of the electrical and optical stimulation units 210, 220 according to the selected time and frequency programs. After the expiration of the stimulation time, an alarm signal is emitted from the acoustic alarm device 460. The treatment apparatuses 300 are separated from the body surface 1. Subsequently, the adhering components 110 of the positioning devices 100 are discarded, while the stimulation devices 200 are prepared for further use, e. g. using the recharging device 500.

The features of the invention in the above description, the drawings and the claims can be of significance individually or in combination or in sub-combination for the realization for the invention in its various embodiments.

1. A positioning device, which is configured for positioning a stimulation device, in particular for low level laser therapy applications, on a body surface of a person to be stimulated, comprising:
an adhering component, which is adapted for accommodating the stimulation device and for a releasable arrangement on the body surface,
an antenna device, which is fixedly connected with the adhering component, said antenna device being arranged for receiving control signals for controlling the stimulation device,
a carrier contact section, which is fixedly connected with the adhering component, said carrier contact section being arranged for providing an electrical contact with the stimulation device, and
a switching device, which is fixedly connected with the adhering component and electrically interconnected between the antenna device and the carrier contact section.

2. The positioning device according to claim 1, wherein the adhering component comprises
at least one adhesive layer being arranged for contact with the body surface, and
a carrier web, which carries the antenna device and the switching device and further is adapted to form a receptacle for accommodating the stimulation device.

3. The positioning device according to claim 2, wherein
the antenna device and the switching device are arranged on an outer surface of the carrier web, and
the carrier contact section is arranged on an inner side of the receptacle provided by the carrier web.

4. The positioning device according to claim 1, wherein
when the stimulation device is coupled with the positioning device, a length of an electrical connection between the antenna device and the switching device is shorter than a length of an electrical connection between the switching device via the carrier contact section to the stimulation device.

5. The positioning device according to claim 1, wherein
the carrier contact section includes two contacts, and
the switching device is adapted for electrically connecting the contacts in response to a control signal received by the antenna device.

6. The positioning device according to claim 1, wherein
the adhering component is a disposable component having a shape of an adhesive plaster.

7. A treatment apparatus, which is configured for a stimulation treatment of a person, comprising:
the positioning device according to claim 1, and
a stimulation device comprising at least one stimulation unit, a power supply section for supplying electrical power to the at least one stimulation unit, and a stimulation device contact section, wherein
the stimulation device is coupled with the positioning device, and
the stimulation device contact section is in contact with the carrier contact section of the positioning device.

8. The treatment apparatus according to claim 7, wherein
the at least one stimulation unit includes at least one of an optical stimulation unit and an electrical stimulation unit.

9. The treatment apparatus according to claim 7, wherein
the stimulation device comprises an enclosure accommodating the at least one stimulation unit and the power supply, wherein
the enclosure includes the stimulation device contact section, which is arranged for a contact with the carrier contact section of the positioning device.

10. The treatment apparatus according to claim 7, further comprising
an anchoring section of the stimulation device, wherein the anchoring section is adapted for a releasable connection of the stimulation device with the positioning device.

11. The treatment apparatus according to claim 10, wherein the anchoring section includes at least one of the features
the anchoring section comprises spikes, which are arranged for a connection with the carrier web,
the anchoring section comprises receptacle grooves for accommodating edges of the carrier web, and
the anchoring section is provided on an outer surface of the enclosure.

12. A stimulation system, comprising
a plurality of treatment apparatuses according to claim 7, and
a main control, which is adapted for creating control signals for controlling the treatment apparatuses.

13. The stimulation system according to claim 12, wherein
the main control further includes interface sections for electrically coupling and recharging the stimulation devices of the treatment apparatuses.

14. A stimulation device, comprising:
at least one stimulation unit,
a power supply section for supplying electrical power to the at least one stimulation unit, and
a stimulation device contact section being exposed on an outer surface of the stimulation device.

15. A stimulation method of electro-optically stimulating a body of a person to be stimulated, comprising the steps of
providing the stimulation system of claim 12 including the plurality of treatment apparatuses,
positioning the treatment apparatuses on the body surface, and
activating the treatment apparatuses by creating control signals for controlling the treatment apparatuses using the main control, wherein the at least one stimulation unit of each stimulation device is switched with the switching device of the associated positioning device.
